(19) United States
(12) Patent Application Publication
(10) Pub. No.: US 2004/0152971 A1
(43) Pub. Date: Aug. 5, 2004

(54) Optimal k-needle placement strategy considering an approximate initial needle position

(76) Inventor: Markus Kukuk, Palo Alto, CA (US)

Correspondence Address: Siemens Corporation, Intellectual Property Department, 170 Wood Avenue South, Iselin, NJ, 08830, US

(21) Appl. No.: 10/357,112

(22) Filed: Feb. 3, 2003

(51) Int. Cl. A61B 5/05

(52) U.S. Cl. 600/424

(57) ABSTRACT

Techniques are provided to determine, for a given set of possible initial needle positions, the smallest set of needles needed to guarantee a successful biopsy. Advantageously, this problem may be formulated as a “Set Covering Problem” (SCP), a well-known combinatorial optimization problem for which good approximations are known. Additionally, the present invention provides techniques to maximize the coverage of the possible initial positions for a given maximum number of k needles. This aspect of the invention may be formulated as a “Maximum k-Coverage Problem”.

### FIELD OF THE INVENTION

The present invention relates generally to accurate positioning of a surgical instrument and, more particularly, to techniques for determining an optimal k-needle placement strategy given an approximate initial needle position.

### BACKGROUND OF THE INVENTION

A needle biopsy is a minimally invasive surgical procedure, often used in the diagnosis and staging of cancer patients. In general, the goal is to take a sample of a suspicious tissue (target) by placing a biopsy needle inside the target. Since the target is often not directly visible to the physician, numerous methods for guiding biopsies have been developed. Procedures that have attracted special attention in recent years include biopsy of the prostrate, breast, liver and lung.

In many cases it is common practice to take more than one tissue sample, in order to increase the probability of hitting the target. Instead of using a simple trial-and-error approach, biopsy strategies have been developed, among others for prostrate cancer biopsies. The “k-Needle Placement Strategy” is a biopsy protocol that specifies how to place k (biopsy) needles, such that the probability of success is maximized. The placement of a needle may be specified by an instrument parameter vector. This represents a suitable parameterization of its degrees of freedom, e.g., by two angles and an insertion depth.

In a special class of biopsy problems, the initial needle position is known only approximately. A typical example for such a procedure is a transbronchial needle aspiration biopsy (TBNA). FIG. 1 illustrates a bronchoscope 101 being used to perform a TBNA. This procedure is performed by maneuvering the bronchoscope 101 to a suitable site within a patient's tracheobronchial tree 102. Then a bronchoscopist inserts a needle through the bronchoscope 101 and punctures the bronchial wall in order to hit the target 103 behind.

Conventional methods to guide TBNAs are based on estimating the position and orientation of the bronchoscope's tip. For example, Solomon et al., “Real-time Bronchoscope Tip Localization Enables Three-dimensional CT Image Guidance for Transbronchial Needle Aspiration in Swine,” CHEST '98, vol. 114/5, pp. 1405-1410, describes the use of position sensors inside the bronchoscope's tip. Mori et al., “A Method for Tracking the Camera Motion of Real Endoscope by Epipolar Geometry Analysis and Virtual Endoscopy System,” MICCAI, vol. 2208 of LNCS, Spring 2001, pp. 1-8, describes a technique for analyzing video images from a CCD camera inside the bronchoscope's tip to achieve a continuous tracking.

### SUMMARY OF THE INVENTION

In various embodiments of the present invention, there is provided a method for determining an optimal instrument placement strategy. The method for determining an optimal instrument placement strategy includes the steps of determining a set of initial instrument positions, and finding the smallest set of instrument placement parameter vectors that fully cover the set of initial instrument positions. The better the coverage of the initial instrument positions, the higher the probability of success for the procedure, e.g., biopsy.

The set of initial instrument positions may be a set of initial needle positions. The set of instrument placement parameter vectors may be used to define the placements of a needle.

The method can be formulated as a “Set Covering Problem” (SCP), a well-known combinatorial optimization problem.

The method may further include the step of ordering the smallest set of instrument placement parameter vectors in order of decreasing success probability. The method can include outputting these instrument placement parameter vectors (e.g., on a screen or a report).

In various other embodiments of the present invention, a method is provided for determining an optimal instrument placement strategy that includes the steps of determining a set of initial instrument positions, and finding for a given number k, a set of k instrument placement parameter vectors that provide maximum coverage for the predetermined set of initial instrument positions. The given number k may be a maximum number of needles that can be placed without putting the patient's safety at risk. The method may be formulated as a “Maximum k-Coverage Problem” (kCP). The ordered set of instrument placement parameter vectors may be outputted.

### BRIEF DESCRIPTION OF THE DRAWINGS

FIG. 1 illustrates an example of a transbronchial needle aspiration (TBNA) biopsy;

FIG. 2 is a block diagram of a computer processing system to which the present invention may be applied;

FIG. 3 illustrates an example of a set of possible endoscope positions divided into k subsets, each subset representing endoscope positions covered by a particular needle parameter;

FIG. 4(a) illustrates a scan S^{T}^{^{Δ}} (p₁) from viewpoint p₁;

FIG. 4(b) illustrates three scans from viewpoints p₁,p₂,p₃ with only one cell (boxed) covered by all three scans; and

FIG. 5 illustrates an exemplary report showing success probabilities for k needle placements.

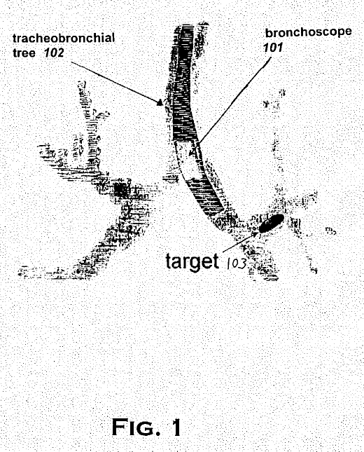

### DESCRIPTION OF PREFERRED EMBODIMENTS

To facilitate a clear understanding of the present invention, illustrative examples are provided herein which describe the invention in applications directed to biopsy procedures. However, the invention is not solely limited to applications related to biopsy procedures. It is to be appreciated that the invention may also be used to determine an optimal strategy for placing other types of surgical instruments, in cases where the target area is known but the initial position of the surgical instrument is given with some error.

It is also to be understood that the present invention may be implemented in various forms of hardware, software, firmware, special purpose processors, or a combination thereof. Preferably, the present invention is implemented in software as a program tangibly embodied on a program storage device.

The program may be uploaded to, and executed by, a machine comprising any suitable architecture. Preferably, the machine is implemented on a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the program (or combination thereof) which is executed via the operating system. In addition, various other peripheral devices may be connected to the computer platform such as an additional data storage device and a printing device.

It is to be understood that, because some of the constituent system components and method steps depicted in the accompanying figures are preferably implemented in software, the actual connections between the system components (or the process steps) may differ depending upon the manner in which the present invention is programmed.

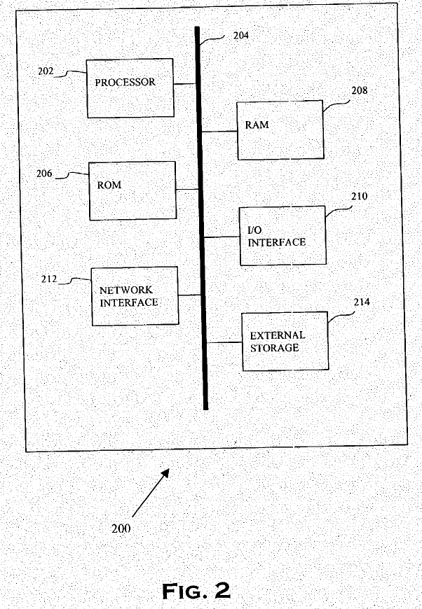

FIG. 2 is a block diagram of a computer processing system 200 to which the present invention may be applied according to an embodiment of the present invention. The system 200 includes at least one processor (hereinafter processor) 202 operatively coupled to other components via a system bus 204. A read-only memory (ROM) 206, a random access memory (RAM) 208, an I/O interface 210, a network interface 212, and external storage 214 are operatively coupled to the system bus 204. Various peripheral devices such as, for example, a display device, a disk storage device (e.g., a magnetic or optical disk storage device), a keyboard, and a mouse, may be operatively coupled to the system bus 204 by the I/O interface 210 or the network interface 212.

Those skilled in the art will appreciate that other alternative computing environments may be used without departing from the spirit and scope of the present invention.

In general, the present invention provides techniques to determine, for a given set of possible initial needle positions, the smallest set of needles that fully cover the given set of initial needle positions. Additionally, the present invention provides techniques to maximize the coverage of the possible initial positions for a given maximum number of k needles. The advantage of considering this variation of the problem is that there exists an approximate solution, which is easy to implement and is guaranteed to be within a factor 1−1/e of the exact solution.

The basic idea is to find needles that “cover” as much of the approximate area as possible. A needle covers an area, if for any position within this area the needle in question hits the target. One goal is to solve the problem by minimizing the number of needles for a full coverage. We formulate the problem of finding the smallest set of needles that cover all initial positions as the problem of finding the minimum set cover in the needle parameter domain. This problem in turn, can be directly formulated as the “Set Covering Problem”, a well-known NP-hard optimization problem.

Another important goal is to maximize the coverage of the possible initial positions for a given maximum number of k needles. We formulate this problem as the “Maximum k-Coverage Problem” (kCP), likewise a well known NP-hard, general optimization problem.

Assumptions

The problem addressed herein is based on the following three assumptions:

Let α, β, and γ be positive integers.

1. The initial position domain P⊂R^{α} a set of possible initial locations for the needle, before placement. The real, but unknown initial placement \tilde{p}εP does not change for the time the k needles are placed.

2. The target domain T⊂R^{β}.

3. A function f:P×T→N, which computes for a given pεP and tεT the necessary needle parameter nεN to hit t from position p. We denote N⊂R^{γ} as the “needle parameter domain”. We also introduce the dual function \overline{f}:P×N→R^{β} that computes for a given position p and a needle parameter n the resulting needle tip position.

Note that the codomain of \overline{f} is R^{β}, because:

for p≠q:(n=f(p,tεT))→(\overline{f}(q,n)εT),

p,qεP,

tεT

Given these assumptions, the k-needle placement problem is to determine a set N_{*}⊂N of k needle parameters, such that P is covered as well as possible. Three such sets N_{*} and their corresponding sets P_{*} in the position domain P are considered:

Definition (N_{*} and P_{*}). Let

Nₙₐᵢᵥₑ,N_{better},Nₒₚₜ

be subsets of the needle parameter domain N. Then the corresponding sets in the position domain P are denoted by

Pₙₐᵢᵥₑ,P_{better},Pₒₚₜ

and defined as:

P_{*}={pεP|\overline{f}(p,n) is an element of T, nεN_{*}}

Set P_{*} is the set of all pεP that are mapped into the target by a needle of set N_{*}.

Naïve Method

A naive method to find k needle parameters that cover P, is to firstly select a set P^{Δ} of k samples from P. For each sample pᵢεP^{Δ} a needle parameter is calculated that would bring the needle tip into the center of the target:

Nₙₐᵢᵥₑ=f(P^{Δ}, t_{center}), |P^{Δ}|=k.

Note that this abbreviated notation is used as an equivalent for:

Nₙₐᵢᵥₑ={n=f(pᵢ,t_{center})|pᵢεP^{Δ}}

In other words, Nₙₐᵢᵥₑ is a set of needle parameters that hit the target from at least all positions pεP^{Δ}. It is “hoped” that Nₙₐᵢᵥₑ maps as many pεP into the target as possible.

This “strategy” has at least two shortcomings. The first shortcoming is that P is not necessarily well covered:

P^{Δ}⊂Pₙₐᵢᵥₑ⊂P.

It is not guaranteed that for all endoscope positions pεP there exists a needle in Nₙₐᵢᵥₑ which hits the target. Secondly, Nₙₐᵢᵥₑ is not necessarily minimal. There may exist a set N_{better}⊂N such that

|N_{better}|<|Nₙₐᵢᵥₑ| and P_{better}⊃Pₙₐᵢᵥₑ.

P_{better} covers at least as much of P as Pₙₐᵢᵥₑ, while needing fewer needles. These observations suggest the formulation of the k-needle placement problem as an optimization problem.

An “Optimal Strategy”

An optimal k-needle placement strategy is a set Nₒₚₜ⊂N of needle parameters, such that

1. Pₒₚₜ=P and

2. |Nₒₚₜ|=minimal

In other words, for all endoscope positions pεP there exists a needle in Nₒₚₜ which hits the target and no set smaller than Nₒₚₜ guarantees the same.

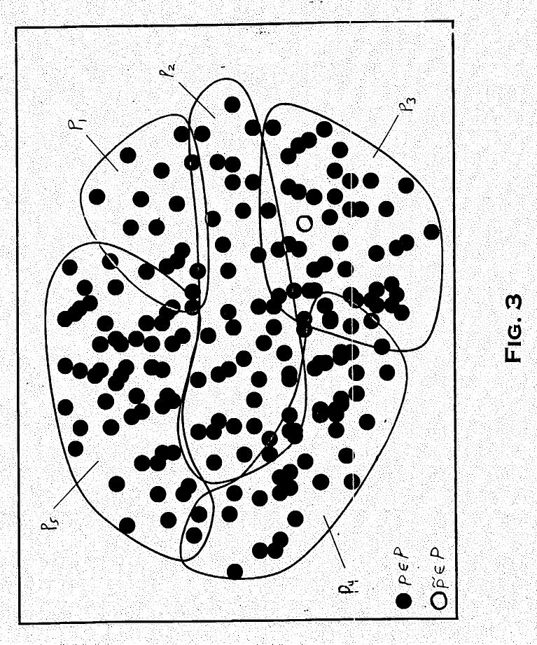

Similar to the definition of N_{*} and P_{*}, let Pᵢ denote the set of all pεP that are mapped into the target by a needle parameter nᵢεNₒₚₜ. The formulation of Nₒₚₜ divides P into a set of k subsets Pᵢ. FIG. 3 shows an example for k=5. It shows five subsets P₁, . . . , P₅, with each Pᵢ induced by a needle parameter nᵢεNₒₚₜ. This example makes it clear that any given real endoscope position \tilde{p} will fall inside a subset Pᵢ and a corresponding needle parameter nᵢ will map \tilde{p} inside the target. Since \tilde{p} is unknown, all five needle parameters have to be tested, one at a time. In this example, the first, second, and fifth needle will fail and the third will hit the target.

Instead of finding the smallest set of subsets in P, we consider a dual problem in the needle parameter domain N. We transform the problem into N by uniformly sampling P and calculating a “scan” of target T from the “perspective” of each sample. The dual problem is then to find a minimum number of points in N such that each scan covers at least one point. This set of points is equivalent to Nₒₚₜ.

Transformation Into the Needle Parameter Domain

Definition: For a given pεP we denote S^{T}(p)⊂N as the “scan” of T from p:

S^{T}(p)=f(p,T).

S^{T}(p) is the set of all needle parameters needed to hit all tεT from p. Position p is called the “viewpoint” of the scan.

We now assume target T is discretized in voxels or cells with side length Δ_{T}. A discretization of T requires as well a discretization of N in the sense that two needle parameters which map a position pεP into the same voxel of T, can be regarded as one needle parameter. This side length of a cell in N follows directly from this interpretation.

Definition: We discretize the needle parameter domain N into cells. The centers of all cells represent the discretized needle parameter domain N^{Δ}. Cell size Δ_{N} is derived from the cell size Δ_{T} in T. Let d( ) be the Euclidean distance:

Δ_{N}=d(n₁,n₂)→max

such that for a pεP:d(\overline{f}(p,n₁),\overline{f}(p,n₂))≦Δ_{T}

We now make the transition from S^{T}(p)⊂N to S^{T}^{^{Δ}}(p)⊂N^{Δ} where S^{T}^{^{Δ}} is the “scan” of T^{Δ} from pεP. The idea is to “round” each nεS^{T}^{^{Δ}} (p) to the center of the cell it falls in. Consequently, it is sufficient for one S^{T}^{^{Δ}}(p) to store for each cell in N^{Δ} either a “1” if the cell contains any element from S^{T}^{^{Δ}}(p) or a “0” if the cell is empty. This yields to the following:

Definition: We denote all cells N^{Δ} as a “cell layer”. Each cell of a cell layer stores two pieces of information: 1. nᵢ^{c}εN^{Δ} the center of cell i. 2. Vᵢ⊂P the set of viewpoints of cell i. The “cell center” is the needle parameter in the center of the cell. Set Vᵢ is the set of viewpoints of all scans that cover cell i.

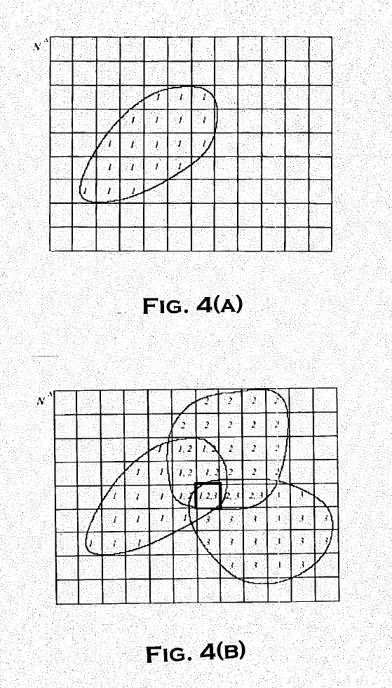

FIG. 4(a) shows N^{Δ} divided into cells and a scan S^{T}^{^{Δ}}(p₁). For each cell the set of viewpoints Vᵢ is given. The set is either {1} (subscript of viewpoint p₁) if the cell is covered by the scan or the empty set { }, if the cell is not covered.

To transform our problem from P to N^{Δ}, we uniformly sample P and calculate for each pᵢεP the scan S^{T}^{^{Δ}}(pᵢ). FIG. 4(b) shows an example for three samples p₁,p₂,p₃εP. This example shows a boxed cell, which is covered by all three scans. With nᵢ^{c} the center of this cell, this can be interpreted as:

\overline{f}(p₁,nᵢ^{c})εT^{Δ} [symbol] \overline{f}(p₂,nᵢ^{c})εT^{Δ} [symbol] \overline{f}(p₃,nᵢ^{c})εT^{Δ}

In other words, only one needle parameter nᵢ^{c} is needed to map all three positions into the target. Positions p₁,p₂,p₃ are members of the same subset, induced by nᵢ^{c}. The goal of dividing P into as few subsets as possible can now be formulated as finding as few cells in N^{Δ}, such that each scan covers at least one selected cell. This problem can be directly reduced to the following “classic” optimization problem.

“Set Covering Problem” and “Maximum k-Coverage Problem”

The “Set Covering Problem” (SCP) is a well-known NP-hard combinatorial optimization problem, which can be formulated as:

Set Covering Problem: A finite set U of elements and a class S of subsets of U is given. Let Sᵢ denote the i-th subset in S.

The task is to select subsets Sᵢ, such that every element in U belongs to at least one Sᵢ. A selection W⊂S with this property is called a set cover of U with respect to S.

The optimization problem is to find a set cover W of minimum cardinality:

SCP(U,S)={W|W| is the set cover of U of minimum cardinality}.

The SCP is subject of numerous publications in the operations research and mathematical literature. Many applications of the set covering problem to real-world problems, such as resource allocation and scheduling have been described. Exact solutions for moderately sized problems using a dual heuristic have been reported. For large problems, approximative schemes have been suggested.

An interesting variation of the SCP is the “Maximum k-Coverage Problem” (kCP). The kCP considers additionally to U and S, an integer k and a weight w(u) for each element of U. The optimization problem is to select k subsets Sᵢ from S such that the weight of the elements in Sᵢ is maximized. It has been shown that the greedy approach to this NP-hard problem, which selects at each stage the subset that gives maximum improvement, is guaranteed to be within a factor of 1−(1−1/k)^{k}>1−1/e of the optimal solution.

Formulation of the Problem as an SCP and kCP

The connection between our problem and the SCP can be established as follows:

Let P^{Δ} be a set of samples of P, Vᵢ⊂P^{Δ} the set of viewpoints of cell i and W an arbitrary minimal set cover:

WεSCP(U,S),

where

U=P^{Δ},S={V₁, V₂, . . . , V_{|N}_{^{Δ}}_{|}}

Let nᵢ^{c}εN^{Δ} be the needle parameter in the center of cell i. Then an optimal k-Needle placement strategy is given by:

Nₒₚₜ={nᵢ^{c}|VᵢεW}.

The Pₒₚₜ=P condition follows from the SCP condition that every element in U belongs to at least one selected subset Sᵢ. The |Nₒₚₜ|=minimal condition follows from the minimization of the sets covers' cardinality.

For example, given the situation shown in FIG. 4(b), U={p₁,p₂,p₃}, S={{ },{p₁},{p₂},{p₃},{p₁,p₂},{p₂,p₃},{p₁,p₂,p₃}}, W={{p₁,p₂,p₃}} and Nₒₚₜ={nᵢ^{c}}, where i is the boxed cell.

With this formulation, a subset of P, induced by a nᵢ^{c}εNₒₚₜ is given by Vᵢ. It is important to note that the quality of solution Nₒₚₜ depends on the sample density of P^{Δ}.

The connection between our problem and the kCP follows directly from the above theorem, with the weight function given by: w(u)=1, for all uεU. This weight function favors cells that are covered by many scans, since the kCP maximizes the sum of the weights of al elements of all selected subsets.

The kCP is an interesting approach to our problem because of two reasons: Firstly, the greedy approach is easy to implement, by simply selecting at each stage the cell with the highest cardinality of Vᵢ and subsequently updating all Vᵢ. Secondly, it has been shown, for small k, a greedily constructed solution is within an acceptable factor from the exact solution. For example, for k=2, the factor is 0.75.

The needle parameters given by Nₒₚₜ should be executed in the order of decreasing success probability. Regarding a chosen sample density, the probability of hitting target T with a needle parameter nᵢ^{c}εNₒₚₜ is given by

$$\frac{\vert {V}_{i} \vert}{\vert {P}^{Δ} \vert} .$$

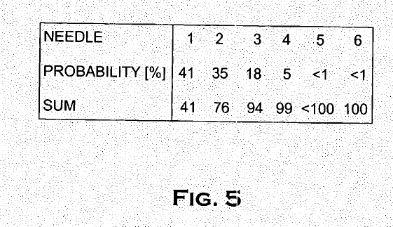

We have presented an optimal strategy for placing k biopsy needles given a large number of possible needle positions. Besides the actual needle parameters, we provide a table to the physician, which contains a probability of success for each needle. Referring to FIG. 5, an exemplary table shows that two needles cover 76% and three needles 94% of all initial positions. By placing the needles in order of decreasing probability, the physician can decide after each needle, whether the gain in the overall probability of success by employing the next needle outweighs the risk to the patient. Overall, this approach provides valuable decision support to the physician regarding how many needles to place and how to place them. For example, depending on the concrete condition of the patient, a decision can be made whether a third or even fourth needle is advisable. Based on this table, the gain of five or more needles is negligible.

While the above description of the present invention refers to the use of known techniques for solving the “Set Covering Problem” (SCP) and the “Maximum k-Coverage Problem” (kCP), it should be appreciated that any known or later developed algorithm for solving these types of problems may be employed without departing from the spirit and scope of the present invention.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for determining an optimal instrument placement, comprising the steps of:
determining a set of initial instrument positions; and
finding the smallest set of instrument placement parameter vectors that guarantees a successful procedure for any initial instrument position in the set of initial instrument positions.

2. The method of claim 1, wherein the procedure relates to hitting a target with a needle.

3. The method of claim 1, wherein the set of initial instrument positions is a set of initial needle positions.

4. The method of claim 3, wherein the set of instrument placement parameter vectors define the placements of a needle.

5. The method of claim 1, wherein the finding step is formulated as a Set Covering Problem (SCP).

6. The method of claim 1, further comprising the step of ordering the smallest set of instrument placement parameter vectors in order of decreasing success probability.

7. The method of claim 6, further including the step of outputting the ordered smallest set of instrument placement parameter vectors.

8. A method for determining an optimal instrument placement, comprising the steps of:
determining a set of initial instrument positions; and
finding, for a predetermined number k, a set of k instrument placement parameter vectors that provide maximum coverage for the predetermined set of initial instrument positions.

9. The method of claim 8, further including the step of receiving k as an input parameter.

10. The method of claim 8, wherein the finding step is formulated as a Maximum k-Coverage Problem (kCP).

11. The method of claim 8, further comprising the step of ordering the set of instrument placement parameter vectors in order of decreasing success probability.

12. The method of claim 11, further including the step of outputting the ordered set of instrument placement parameter vectors.

13. A program storage device readable by a machine, tangibly embodying a program of instructions executable on the machine to perform method steps for determining an optimal instrument placement, comprising the method steps of:
determining a set of initial instrument positions; and
finding the smallest set of instrument placement parameter vectors that guarantees a successful procedure for any initial instrument position in the set of initial instrument positions.

14. A program storage device readable by a machine, tangibly embodying a program of instructions executable on the machine to perform method steps for determining an optimal instrument placement, comprising the method steps of:
determining a set of initial instrument positions; and
finding, for a predetermined number k, a set of k instrument placement parameter vectors that provide maximum coverage for the predetermined set of initial instrument positions.
